(19) United States
(12) Patent Application Publication
(10) Pub. No.: US 2006/0270730 A1
(43) Pub. Date: Nov. 30, 2006

(54) Histone deacetylase inhibitors as immunosuppressants

(76) Inventor: Andreas Katopodis, Oberwill (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/567,515

(22) PCT Filed: Aug. 6, 2004
(86) PCT No.: PCT/EP04/08849

§ 371 Date: Feb. 7, 2006

Related U.S. Application Data
(60) Provisional application No. 60/493,320, filed on Aug. 7, 2003.

(51) Int. Cl. A61K 31/405; A61K 31/44; A61K 31/4745; A61K 38/12; A61K 38/13; A61K 39/395; A61K 31/66; A61K 31/395

(52) U.S. Cl. 514/419; 514/575; 514/357; 514/11; 514/291; 514/9; 514/183; 514/109; 424/145.1

(57) ABSTRACT

A method of preventing or suppressing an immune response or immune mediated response comprising administering an effective amount of an histone deacetylase inhibitor compound alone or in combination with a second pharmacologically active agent.

The present invention relates to histone deacetylase (“HDAC”) inhibitor compounds having immunosuppressant activity which are useful as pharmaceuticals, particularly for use as immunosuppressant agents for delaying, preventing, or treating acute or chronic transplant rejection.

### BACKGROUND OF THE INVENTION

Organ transplants of liver, kidney, lung and heart are now regularly performed as treatment for endstage organ disease. Allograft as well as xenograft transplants have been performed. However, because of problems with acute rejection as well as long-term chronic rejection, organ transplantation is not yet a permanent solution to irreversible organ disease.

Acute rejection manifests itself as acute graft dysfunction and is the result of an immune reaction of the recipient against the donor tissue. Acute rejection can lead to graft loss if not treated. Increased incidence of acute rejection has been correlated with increased danger for chronic rejection. Chronic rejection, which manifests as progressive and irreversible graft dysfunction, is the leading cause of organ transplant loss. Chronic rejection appears to be inexorable and uncontrollable because there is no known effective treatment or prevention modality. Thus, there continues to exist a need for an immunosuppressant treatment effective e.g. in preventing, controlling or reversing manifestations of acute and chronic graft rejection.

Reversible acetylation of histones is a major regulator of gene expression that acts by altering accessibility of transcription factors to DNA. In normal cells, histone deacetylase (HDAC) and histone acetyltransferase together control the level of acetylation of histones to maintain a balance.

Mammalian HDACs can be divided into three classes according to sequence homology: Class I consists of the yeast Rpd3-like proteins (HDAC 1, 2, 3, 8 and 11). Class II consists of the yeast HDA1-like proteins (HDAC 4, 5, 6, 7, 9, and 10). Class II comprises the yeast SIR2-like proteins. (SIRT 1, 2, 3, 4, 5, 6, 7).

Inhibitors of histone deacetylase inhibitors induce hyperacetylation of histones that modulate chromatin structure and gene expression. These inhibitors also induce growth arrest, cell differentiation, and apoptosis of tumor cells.

Inhibition of HDAC results in a variety of cellular responses. Surprisingly, it has now been found that HDAC inhibitor compounds, alone or in combination with other therapeutic agents, are effective as immunosuppressants and can be used as anti-transplant rejection drugs or to treat autoimmune or inflammatory diseases.

### SUMMARY OF THE INVENTION

The present invention relates to the use of HDAC inhibitor (“HDAI”) compounds, alone or in combination with other therapeutic agents, such as immunosuppressants or immunomodulators and, more specifically, for the treatment and/or prevention of immune disorders such as autoimmune or inflammatory diseases. HDAI compounds are also used to promote the viability of transplanted material, and for delaying, preventing, or treating acute or chronic transplant rejection.

More particularly, the invention relates to inhibiting graft rejection (e.g. acute or chronic graft rejection) by administering to a graft recipient a therapeutically effective amount of an HDAI compound either as single agents or in combination with other immunosuppressants or immunomodulators.

In one embodiment, this invention discloses a method for suppressing an immune response of a subject, preferably an animal, more preferably a human, by administering to the animal an effective amount of an HDAI compound or combination.

Further embodiments include compositions containing an HDAI compound, alone or in combination with other therapeutic agents such as other immunosuppressants or immunomodulators. The composition may contain an HDAI compound of formula I alone or in combination with other therapeutic agents such as other immunosuppressants or immunomodulators, and pharmaceutically acceptable carriers.

Most particularly, co-administration of an HDAI and an second pharmaceutically active agent such as an immunomodulator or an immunosuppressant, result in a synergistic-effect which effect is greater than the sum of the effect achieved for the either compound separately.

### DETAILED DESCRIPTION OF THE INVENTION

The present invention relates to the use of histone deacetylase inhibitor compounds as immunosuppressants or immunomodulators and, more specifically, for the treatment and/or prevention of an immune disorder such as autoimmune or inflammatory diseases. The HDAI compounds of the present invention are also used to promote the viability of transplanted material, and for reducing or prevention organ or tissue transplant rejection.

More particularly, the invention relates to inhibiting graft rejection (e.g. acute or chronic graft rejection) by administering to a graft recipient a therapeutically effective amount of an HDAI alone or in combination with other therapeutic agents such as other immunosuppressants or immunomodulators. The present invention further relates to preventing or treating graft-versus-host diseases such as following bone marrow transplant.

The acetylation and deacetylation of histones of nucleosome core proteins play an important role in the regulation of gene expression. Histone deacetylation determines the transcriptional suppression of these genes resulting in growth stimulation. Histone deacetylases, HDACs, also play a role in modeling the structure of chromatin. These enzymes have been shown to regulate gene expression by deacetylating transcription factors such as p53 and to participate in cell cycle regulation. Inhibition of histone deacetylase results in a variety of cellular responses.

In one embodiment, this invention discloses a method for treating, preventing or suppressing an immune disorder, an immune response or an immune mediated response of an animal by administering to the animal an effective amount of an HDAI compound.

The present invention is directed to any compound which acts as an inhibitor of histone deacetylase, preferably inhibiting the mixed lymphocyte reaction (MLR), most preferably having an IC50 of <500 nM in the mouse or human MLR.

In a preferred embodiment, the HDAI has the following structure (I): 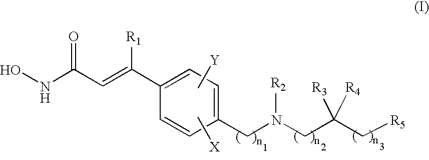
wherein
R₁ is H, halo, or a straight chain C₁-C₆ alkyl (especially methyl, ethyl or n-propyl, which methyl, ethyl and n-propyl substituents are unsubstituted or substituted by one or more substituents described below for alkyl substituents);
R₂ is selected from H, C₁-C₁₀ alkyl, (preferably C₁-C₆ alkyl, e.g. methyl, ethyl or —CH₂CH₂—OH), C₄-C₉ cycloalkyl, C₄-C₉ heterocycloalkyl, C₄-C₉ heterocycloalkylalkyl, cycloalkylalkyl (e.g., cyclopropylmethyl), aryl, heteroaryl, arylalkyl (e.g. benzyl), heteroarylalkyl (e.g. pyridylmethyl), —(CH₂)ₙC(O)R₆, —(CH₂)ₙOC(O)R₆, amino acyl, HON—C(O)—CH═C(R₁)-aryl-alkyl- and —(CH₂)ₙR₇;
R₃ and R₄ are the same or different and independently H, C₁-C₆ alkyl, acyl or acylamino, or R₃ and R₄ together with the carbon to which they are bound represent C═O, C═S, or C═NR₈, or R₂ together with the nitrogen to which it is bound and R₃ together with the carbon to which it is bound can form a C₄-C₉ heterocycloalkyl, a heteroaryl, a polyheteroaryl, a non-aromatic polyheterocycle, or a mixed aryl and non-aryl polyheterocycle ring;
R₅ is selected from H, C₁-C₆ alkyl, C₄-C₉ cycloalkyl, C₄-C₉ heterocycloalkyl, acyl, aryl, heteroaryl, arylalkyl (e.g. benzyl), heteroarylalkyl (e.g. pyridylmethyl), aromatic polycycles, non-aromatic polycycles, mixed aryl and non-aryl polycycles, polyheteroaryl, non-aromatic polyheterocycles, and mixed aryl and non-aryl polyheterocycles;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H, halo, C₁-C₄ alkyl, such as CH₃ and CF₃, NO₂, C(O)R₁, OR₉, SR₉, CN, and NR₁₀R₁₁;
R₆ is selected from H, C₁-C₆ alkyl, C₄-C₉ cycloalkyl, C₄-C₉ heterocycloalkyl, cycloalkylalkyl (e.g., cyclopropylmethyl), aryl, heteroaryl, arylalkyl (e.g., benzyl, 2-phenylethenyl), heteroarylalkyl (e.g., pyridylmethyl), OR₁₂, and NR₁₃R₁₄;
R₇ is selected from OR₁₅, SR₁₅, S(O)R₁₆, SO₂R₁₇, NR₁₃R₁₄, and NR₁₂SO₂R₆;
R₈ is selected from H, OR₁₅, NR₁₃R₁₄, C₁-C₆ alkyl, C₄-C₉ cycloalkyl, C₄-C₉ heterocycloalkyl, aryl, heteroaryl, arylalkyl (e.g., benzyl), and heteroarylalkyl (e.g., pyridylmethyl);
R₉ is selected from C₁-C₄ alkyl, for example, CH₃ and CF₃, C(O)-alkyl, for example C(O)CH₃, and C(O)CF₃;
R₁₀ and R₁₁ are the same or different and independently selected from H, C₁-C₄ alkyl, and —C(O)-alkyl;
R₁₂ is selected from H, C₁-C₆ alkyl, C₄-C₉ cycloalkyl, C₄-C₉ heterocycloalkyl, C₄-C₉ heterocycloalkylalkyl, aryl, mixed aryl and non-aryl polycycle, heteroaryl, arylalkyl (e.g., benzyl), and heteroarylalkyl (e.g., pyridylmethyl);
R₁₃ and R₁₄ are the same or different and independently selected from H, C₁-C₆ alkyl, C₄-C₉ cycloalkyl, C₄-C₉ heterocycloalkyl, aryl, heteroaryl, arylalkyl (e.g., benzyl), heteroarylalkyl (e.g., pyridylmethyl), amino acyl, or R₁₃ and R₁₄ together with the nitrogen to which they are bound are C₄-C₉ heterocycloalkyl, heteroaryl, polyheteroaryl, non-aromatic polyheterocycle or mixed aryl and non-aryl polyheterocycle;
R₁₅ is selected from H, C₁-C₆ alkyl, C₄-C₉ cycloalkyl, C₄-C₉ heterocycloalkyl, aryl, heteroaryl, arylalkyl, heteroarylalkyl and (CH₂)ₘZR₁₂;
R₁₆ is selected from C₁-C₆ alkyl, C₄-C₉ cycloalkyl, C₄-C₉ heterocycloalkyl, aryl, heteroaryl, polyheteroaryl, arylalkyl, heteroarylalkyl and (CH₂)ₘZR₁₂;
R₁₇ is selected from C₁-C₆ alkyl, C₄-C₉ cycloalkyl, C₄-C₉ heterocycloalkyl, aryl, aromatic polycycles, heteroaryl, arylalkyl, heteroarylalkyl, polyheteroaryl and NR₁₃R₁₄;
m is an integer selected from 0 to 6; and
Z is selected from O, NR₁₃, S and S(O),
   or a pharmaceutically acceptable salt thereof.

As appropriate, unsubstituted means that there is no substituent or that the only substituents are hydrogen.

Halo substituents are selected from fluoro, chloro, bromo and iodo, preferably fluoro or chloro.

Alkyl substituents include straight and branched C₁-C₆alkyl, unless otherwise noted. Examples of suitable straight and branched C₁-C₆alkyl substituents include methyl, ethyl, n-propyl, 2-propyl, n-butyl, sec-butyl, t-butyl, and the like. Unless otherwise noted, the alkyl substituents include both unsubstituted alkyl groups and alkyl groups that are substituted by one or more suitable substituents, including unsaturation (i.e. there are one or more double of triple C—C bonds), acyl, cycloalkyl, halo, oxyalkyl, alkylamino, aminoalkyl, acylamino and OR₁₅, for example, alkoxy. Preferred substituents for alkyl groups include halo, hydroxy, alkoxy, oxyalkyl, alkylamino, and aminoalkyl.

Cycloalkyl substituents include C₃-C₉ cycloalkyl groups, such as cyclopropyl, cyclobutyl, cyclopentyl, cyclohexyl and the like, unless otherwise specified. Unless otherwise noted, cycloalkyl substituents include both unsubstituted cycloalkyl groups and cycloalkyl groups that are substituted by one or more suitable substituents, including C₁-C₆ alkyl, halo, hydroxy, aminoalkyl, oxyalkyl, alkylamino, and OR₁₅, such as alkoxy. Preferred substituents for cycloalkyl groups include halo, hydroxy, alkoxy, oxyalkyl, alkylamino and aminoalkyl.

The above discussion of alkyl and cycloalkyl substituents also applies to the alkyl portions of other substituents, such as without limitation, alkoxy, alkyl amines, alkyl ketones, arylalkyl, heteroarylalkyl, alkylsulfonyl and alkyl ester substituents and the like.

Heterocycloalkyl substituents include 3 to 9 membered aliphatic rings, such as 4 to 7 membered aliphatic rings, containing from one to three heteroatoms selected from nitrogen, sulfur and oxygen. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, and 1,4-oxathiapane. Unless otherwise noted, the rings are unsubstituted or substituted on the carbon atoms by one or more suitable substituents, including C₁-C₆ alkyl, C₄-C₉ cycloalkyl, aryl, heteroaryl, arylalkyl (e.g., benzyl), and heteroarylalkyl (e.g., pyridylmethyl), halo, amino, alkyl amino and OR₁₅, for example alkoxy. Unless otherwise noted, nitrogen heteroatoms are unsubstituted or substituted by H, C₁-C₄ alkyl, arylalkyl (e.g., benzyl), and heteroarylalkyl (e.g., pyridylmethyl), acyl, aminoacyl, alkylsulfonyl, and arylsulfonyl.

Cycloalkylalkyl substituents include compounds of the formula —(CH₂)ₙ₅-cycloalkyl wherein n5 is a number from 1-6. Suitable cycloalkylalkyl substituents include cyclopentylmethyl-, cyclopentylethyl, cyclohexylmethyl and the like. Such substituents are unsubstituted or substituted in the alkyl portion or in the cycloalkyl portion by a suitable substituent, including those listed above for alkyl and cycloalkyl.

Aryl substituents include unsubstituted phenyl and phenyl substituted by one or more suitable substituents, including C₁-C₆ alkyl, cycloalkylalkyl (e.g., cyclopropylmethyl), O(CO)alkyl, oxyalkyl, halo, nitro, amino, alkylamino, aminoalkyl, alkyl ketones, nitrile, carboxyalkyl, alkylsulfonyl, aminosulfonyl, arylsulfonyl, and OR₁₅, such as alkoxy. Preferred substituents include including C₁-C₆ alkyl, cycloalkyl (e.g., cyclopropylmethyl), alkoxy, oxyalkyl, halo, nitro, amino, alkylamino, aminoalkyl, alkyl ketones, nitrile, carboxyalkyl, alkylsulfonyl, arylsulfonyl, and aminosulfonyl. Examples of suitable aryl groups include C₁-C₄alkylphenyl, C₁-C₄alkoxyphenyl, trifluoromethylphenyl, methoxyphenyl, hydroxyethylphenyl, dimethylaminophenyl, aminopropylphenyl, carbethoxyphenyl, methanesulfonylphenyl and tolylsulfonylphenyl.

Aromatic polycycles include naphthyl, and naphthyl substituted by one or more suitable substituents, including C₁-C₆ alkyl, cycloalkylalkyl (e.g., cyclopropylmethyl), oxyalkyl, halo, nitro, amino, alkylamino, aminoalkyl, alkylketones, nitrile, carboxyalkyl, alkylsulfonyl, arylsulfonyl, aminosulfonyl and OR₁₅, such as alkoxy.

Heteroaryl substituents include compounds with a 5 to 7 member aromatic ring containing one or more heteroatoms, for example from 1 to 4 heteroatoms, selected from N, O and S. Typical heteroaryl substituents include furyl, thienyl, pyrrole, pyrazole, triazole, thiazole, oxazole, pyridine, pyrimidine, isoxazolyl, pyrazine and the like. Unless otherwise noted, heteroaryl substituents are unsubstituted or substituted on a carbon atom by one or more suitable substituents, including alkyl, the alkyl substituents identified above, and another heteroaryl substituent. Nitrogen atoms are unsubstituted or substituted, for example by R₁₃; especially useful N substituents include H, C₁-C₄ alkyl, acyl, aminoacyl, and sulfonyl.

Arylalkyl substituents include groups of the formula —(CH₂)ₙ₅-aryl, —(CH₂)ₙ₅₋₁-(CHaryl)-(CH₂)ₙ₅-aryl or —(CH₂)ₙ₅₋₁CH(aryl)(aryl) wherein aryl and n5 are as defined above. Such arylalkyl substituents include benzyl, 2-phenylethyl, 1-phenylethyl, tolyl-3-propyl, 2-phenylpropyl, diphenylmethyl, 2-diphenylethyl, 5,5-dimethyl-3-phenylpentyl and the like. Arylalkyl substituents are unsubstituted or substituted in the alkyl moiety or the aryl moiety or both as described above for alkyl and aryl substituents.

Heteroarylalkyl substituents include groups of the formula —(CH₂)ₙ₅-heteroaryl wherein heteroaryl and n5 are as defined above and the bridging group is linked to a carbon or a nitrogen of the heteroaryl portion, such as 2-, 3- or 4-pyridylmethyl, imidazolylmethyl, quinolylethyl, and pyrrolylbutyl. Heteroaryl substituents are unsubstituted or substituted as discussed above for heteroaryl and alkyl substituents.

Amino acyl substituents include groups of the formula —C(O)—(CH₂)ₙ—C(H)(NR₁₃R₁₄)—(CH₂)ₙ—R₅ wherein n, R₁₃, R₁₄ and R₅ are described above. Suitable aminoacyl substituents include natural and non-natural amino acids such as glycinyl, D-tryptophanyl, L-lysinyl, D- or L-homoserinyl, 4-aminobutryic acyl, ±-3-amin-4-hexenoyl.

Non-aromatic polycycle substituents include bicyclic and tricyclic fused ring systems where each ring can be 4-9 membered and each ring can contain zero, 1 or more double and/or triple bonds. Suitable examples of non-aromatic polycycles include decalin, octahydroindene, perhydrobenzocycloheptene, perhydrobenzo-[f]-azulene. Such substituents are unsubstituted or substituted as described above for cycloalkyl groups.

Mixed aryl and non-aryl polycycle substituents include bicyclic and tricyclic fused ring systems where each ring can be 4-9 membered and at least one ring is aromatic. Suitable examples of mixed aryl and non-aryl polycycles include methylenedioxyphenyl, bis-methylenedioxyphenyl, 1,2,3,4-tetrahydronaphthalene, dibenzosuberane, dihdydroanthracene, 9H-fluorene. Such substituents are unsubstituted or substituted by nitro or as described above for cycloalkyl groups.

Polyheteroaryl substituents include bicyclic and tricyclic fused ring systems where each ring can independently be 5 or 6 membered and contain one or more heteroatom, for example, 1, 2, 3, or 4 heteroatoms, chosen from O, N or S such that the fused ring system is aromatic. Suitable examples of polyheteroaryl ring systems include quinoline, isoquinoline, pyridopyrazine, pyrrolopyridine, furopyridine, indole, benzofuran, benzothiofuran, benzindole, benzoxazole, pyrroloquinoline, and the like. Unless otherwise noted, polyheteroaryl substituents are unsubstituted or substituted on a carbon atom by one or more suitable substituents, including alkyl, the alkyl substituents identified above and a substituent of the formula —O—(CH₂CH═CH(CH₃)(CH₂))₁₋₃H. Nitrogen atoms are unsubstituted or substituted, for example by R₁₃; especially useful N substituents include H, C₁-C₄ alkyl, acyl, aminoacyl, and sulfonyl.

Non-aromatic polyheterocyclic substituents include bicyclic and tricyclic fused ring systems where each ring can be 4-9 membered, contain one or more heteroatom, for example, 1, 2, 3, or 4 heteroatoms, chosen from O, N or S and contain zero or one or more C—C double or triple bonds. Suitable examples of non-aromatic polyheterocycles include hexitol, cis-perhydro-cyclohepta[b]pyridinyl, decahydro-benzo[f][1,4]oxazepinyl, 2,8-dioxabicyclo[3.3.0]octane, hexahydro-thieno[3,2-b]thiophene, perhydropyrrolo[3,2-b]pyrrole, perhydronaphthyridine, perhydro-1H-dicyclopenta[b,e]pyran. Unless otherwise noted, non-aromatic polyheterocyclic substituents are unsubstituted or substituted on a carbon atom by one or more substituents, including alkyl and the alkyl substituents identified above. Nitrogen atoms are unsubstituted or substituted, for example, by R₁₃; especially useful N substituents include H, C₁-C₄ alkyl, acyl, aminoacyl, and sulfonyl.

Mixed aryl and non-aryl polyheterocycles substituents include bicyclic and tricyclic fused ring systems where each ring can be 4-9 membered, contain one or more heteroatom chosen from O, N or S, and at least one of the rings must be aromatic. Suitable examples of mixed aryl and non-aryl polyheterocycles include 2,3-dihydroindole, 1,2,3,4-tetrahydroquinoline, 5,11-dihydro-10H-dibenz[b,e][1,4]diazepine, 5H-dibenzo[b,e][1,4]diazepine, 1,2-dihydropyrrolo[3,4-b][1,5]benzodiazepine, 1,5-dihydro-pyrido[2,3-b][1,4]diazepin-4-one, 1,2,3,4,6,11-hexahydro-benzo[b]pyrido[2,3-e][1,4]diazepin-5-one. Unless otherwise noted, mixed aryl and non-aryl polyheterocyclic substituents are unsubstituted or substituted on a carbon atom by one or more suitable substituents, including, —N—OH, ═N—OH, alkyl and the alkyl substituents identified above. Nitrogen atoms are unsubstituted or substituted, for example, by R₁₃; especially useful N substituents include H, C₁-C₄ alkyl, acyl, aminoacyl, and sulfonyl.

Amino substituents include primary, secondary and tertiary amines and in salt form, quaternary amines. Examples of amino substituents include mono- and di-alkylamino, mono- and di-aryl amino, mono- and di-arylalkyl amino, aryl-arylalkylamino, alkyl-arylamino, alkyl-arylalkylamino and the like.

Sulfonyl substituents include alkylsulfonyl and arylsulfonyl, for example methane sulfonyl, benzene sulfonyl, tosyl and the like.

Acyl substituents include groups of the formula —C(O)—W, —OC(O)—W, —C(O)—O—W and —C(O)NR₁₃R₁₄, where W is R₁₆, H or cycloalkylalkyl.

Acylamino substituents include groups of the formula —N(R₁₂)C(O)—W, —N(R₁₂)C(O)—O—W, and —N(R₁₂)C(O)—NHOH and R₁₂ and W are as defined above.

The R₂ substituent HON—C(O)—CH═C(R₁)-aryl-alkyl- is a group of the formula 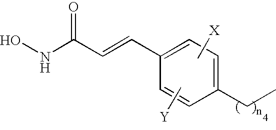
wherein n₄ is 0-3 and X and Y are as defined above.

Preferences for each of the substituents include the following:
R₁ is H, halo, or a straight chain C₁-C₄ alkyl;
R₂ is selected from H, C₁-C₆ alkyl, C₄-C₉ cycloalkyl, C₄-C₉ heterocycloalkyl, cycloalkylalkyl, aryl, heteroaryl, arylalkyl, heteroarylalkyl, —(CH₂)ₙC(O)R₆, amino acyl, and —(CH₂)ₙR₇;
R₃ and R₄ are the same or different and independently selected from H, and C₁-C₆ alkyl, or R₃ and R₄ together with the carbon to which they are bound represent C═O, C═S, or C═NR₈;
R₅ is selected from H, C₁-C₆ alkyl, C₄-C₉ cycloalkyl, C₄-C₉ heterocycloalkyl, aryl, heteroaryl, arylalkyl, heteroarylalkyl, an aromatic polycycle, a non-aromatic polycycle, a mixed aryl and non-aryl polycycle, polyheteroaryl, a non-aromatic polyheterocycle, and a mixed aryl and non-aryl polyheterocycle;
n, n₁, n₂ and n₃ are the same or different and independently selected from 0-6, when n₁ is 1-6, each carbon atom is unsubstituted or independently substituted with R₃ and/or R₄;
X and Y are the same or different and independently selected from H, halo, C₁-C₄ alkyl, CF₃, NO₂, C(O)R₁, OR₉, SR₉, CN, and NR₁₀R₁₁;
R₆ is selected from H, C₁-C₆ alkyl, C₄-C₉ cycloalkyl, C₄-C₉ heterocycloalkyl, cycloalkylalkyl, aryl, heteroaryl, arylalkyl, heteroarylalkyl, OR₁₂, and NR₁₃R₁₄;
R₇ is selected from OR₁₅, SR₁₅, S(O)R₁₆, SO₂R₁₇, NR₁₃R₁₄, and NR₁₂SO₂R₆;
R₈ is selected from H, OR₁₅, NR₁₃R₁₄, C₁-C₆ alkyl, C₄-C₉ cycloalkyl, C₄-C₉ heterocycloalkyl, aryl, heteroaryl, arylalkyl, and heteroarylalkyl;
R₉ is selected from C₁-C₄ alkyl and C(O)-alkyl;
R₁₀ and R₁₁ are the same or different and independently selected from H, C₁-C₄ alkyl, and —C(O)-alkyl;
R₁₂ is selected from H, C₁-C₆ alkyl, C₄-C₉ cycloalkyl, C₄-C₉ heterocycloalkyl, aryl, heteroaryl, arylalkyl, and heteroarylalkyl;
R₁₃ and R₁₄ are the same or different and independently selected from H, C₁-C₆ alkyl, C₄-C₉ cycloalkyl, C₄-C₉ heterocycloalkyl, aryl, heteroaryl, arylalkyl, heteroarylalkyl and amino acyl;
R₁₅ is selected from H, C₁-C₆ alkyl, C₄-C₉ cycloalkyl, C₄-C₉ heterocycloalkyl, aryl, heteroaryl, arylalkyl, heteroarylalkyl and (CH₂)ₘZR₁₂;
R₁₆ is selected from C₁-C₆ alkyl, C₄-C₉ cycloalkyl, C₄-C₉ heterocycloalkyl, aryl, heteroaryl, arylalkyl, heteroarylalkyl and (CH₂)ₘZR₁₂;
R₁₇ is selected from C₁-C₆ alkyl, C₄-C₉ cycloalkyl, C₄-C₉ heterocycloalkyl, aryl, heteroaryl, arylalkyl, heteroarylalkyl and NR₁₃R₁₄;
m is an integer selected from 0 to 6; and
Z is selected from O, NR₁₃, S, S(O).

Useful compounds of the formula I include those wherein each of R₁, X, Y, R₃, and R₄ is H, including those wherein one of n₂ and n₃ is zero and the other is 1, especially those wherein R₂ is H or —CH₂—CH₂—OH.

One suitable genus of hydroxamate compounds are those of formula Ia 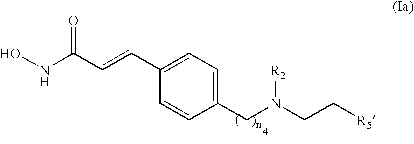
wherein
n₄ is 0-3,
R₂ is selected from H, C₁-C₆ alkyl, C₄-C₉ cycloalkyl, C₄-C₉ heterocycloalkyl, cycloalkylalkyl, aryl, heteroaryl, arylalkyl, heteroarylalkyl, —(CH₂)ₙC(O)R₆, amino acyl and —(CH₂)ₙR₇;
R₅′ is heteroaryl, heteroarylalkyl (e.g., pyridylmethyl), aromatic polycycles, non-aromatic polycycles, mixed aryl and non-aryl polycycles, polyheteroaryl, or mixed aryl and non-aryl polyheterocycles,
   or a pharmaceutically acceptable salt thereof.

Another suitable genus of hydroxamate compounds are those of formula Ia 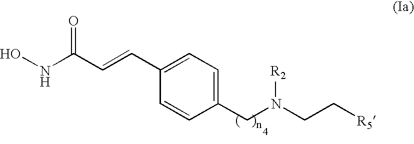
wherein
n₄ is 0-3,
R₂ is selected from H, C₁-C₆ alkyl, C₄-C₉ cycloalkyl, C₄-C₉ heterocycloalkyl, cycloalkylalkyl, aryl, heteroaryl, arylalkyl, heteroarylalkyl, —(CH₂)₂C(O)R₆, amino acyl and —(CH₂)ₙR₇;
R₅′ is aryl, arylalkyl, aromatic polycycles, non-aromatic polycycles, and mixed aryl and non-aryl polycycles; especially aryl, such as p-fluorophenyl, p-chlorophenyl, p-O—C₁-C₄-alkylphenyl, such as p-methoxyphenyl, and p-C₁-C₄-alkylphenyl; and arylalkyl, such as benzyl, ortho, meta or para-fluorobenzyl, ortho, meta or para-chlorobenzyl, ortho, meta or para-mono, di or tri-O—C₁-C₄-alkylbenzyl, such as ortho, meta or para-methoxybenzyl, m,p-diethoxybenzyl, o,m,p-triimethoxybenzyl, and ortho, meta or para- mono, di or tri C₁-C₄-alkylphenyl, such as p-methyl, m,m-diethylphenyl,
   or a pharmaceutically acceptable salt thereof.

Another interesting genus are the compounds of formula Ib 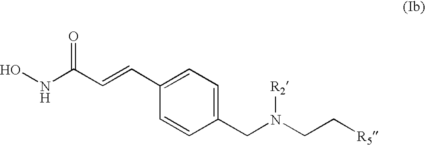
wherein
R₂′ is selected from H, C₁-C₆ alkyl, C₄-C₆ cycloalkyl, cycloalkylalkyl (e.g., cyclopropylmethyl), —(CH₂)₂₋₄OR₂₁ where R₂₁ is H, methyl, ethyl, propyl, and i-propyl, and
R₅″ is unsubstituted 1H-indol-3-yl, benzofuran-3-yl or quinolin-3-yl, or substituted 1H-indol-3-yl, such as 5-fluoro-1H-indol-3-yl or 5-methoxy-1H-indol-3-yl, benzofuran-3-yl or quinolin-3-yl, or a pharmaceutically acceptable salt thereof.

Another interesting genus of hydroxamate compounds are the compounds of formula Ic 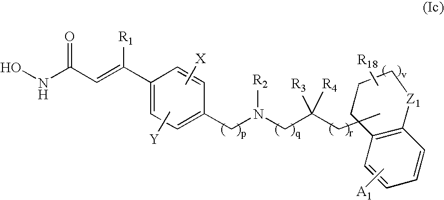
wherein
the ring containing Z₁ is aromatic or non-aromatic, which non-aromatic rings are saturated or unsaturated,
Z₁ is O, S or N—R₂₀,
R₁₈ is H, halo, C₁-C₆alkyl (methyl, ethyl, t-butyl), C₃-C₇cycloalkyl, aryl, for example unsubstituted phenyl or phenyl substituted by 4-OCH₃ or 4-CF₃, or heteroaryl, such as 2-furanyl, 2-thiophenyl or 2-, 3- or 4-pyridyl;
R₂₀ is H, C₁-C₆alkyl, C₁-C₆alkyl-C₃-C₉cycloalkyl (e.g., cyclopropylmethyl), aryl, heteroaryl, arylalkyl (e.g., benzyl), heteroarylalkyl (e.g., pyridylmethyl), acyl (acetyl, propionyl, benzoyl) or sulfonyl (methanesulfonyl, ethanesulfonyl, benzenesulfonyl, toluenesulfonyl);
A₁ is 1, 2 or 3 substituents which are independently H, C₁-C-₆alkyl, —OR₁₉, halo, alkylamino, aminoalkyl, halo, or heteroarylalkyl (e.g., pyridylmethyl),
R₁₉ is selected from H, C₁-C₆alkyl, C₄-C₉cycloalkyl, C₄-C₉heterocycloalkyl, aryl, heteroaryl, arylalkyl (e.g., benzyl), heteroarylalkyl (e.g., pyridylmethyl) and
—(CH₂CH═CH(CH₃)(CH₂))₁₋₃H;
R₂ is selected from H, C₁-C₆ alkyl, C₄-C₉ cycloalkyl, C₄-C₉ heterocycloalkyl,
cycloalkylalkyl, aryl, heteroaryl, arylalkyl, heteroarylalkyl, —(CH₂)ₙC(O)R₆, amino acyl and —(CH₂)ₙR₇;
v is 0, 1 or 2,
p is 0-3, and
q is 1-5 and r is 0 or
q is 0 and r is 1-5,
   or a pharmaceutically acceptable salt thereof. The other variable substituents are as defined above.

Especially useful compounds of formula Ic are those wherein R₂ is H, or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3, especially those wherein Z, is N—R₂₀. Among these compounds R % is preferably H or —CH₂—CH₂—OH and the sum of q and r is preferably 1.

Another interesting genus of hydroxamate compounds are the compounds of formula Id 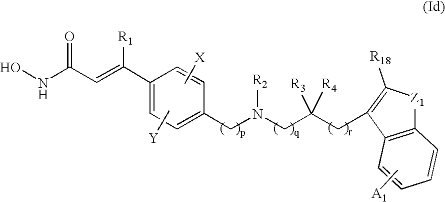
wherein
Z₁ is O, S or N—R₂₀,
R₁₈ is H, halo, C₁-C₆alkyl (methyl, ethyl, t-butyl), C₃-C₇cycloalkyl, aryl, for example, unsubstituted phenyl or phenyl substituted by 4-OCH₃ or 4-CF₃, or heteroaryl,
R₂₀ is H, C₁-C₆alkyl, C₁-C₆alkyl-C₉cycloalkyl (e.g., cyclopropylmethyl), aryl, heteroaryl, arylalkyl (e.g., benzyl), heteroarylalkyl (e.g., pyridylmethyl), acyl (acetyl, propionyl, benzoyl) or sulfonyl (methanesulfonyl, ethanesulfonyl, benzenesulfonyl, toluenesulfonyl);
A₁ is 1, 2 or 3 substituents which are independently H, C₁-C-₆alkyl, —OR₁₉, or halo,
R₁₉ is selected from H, C₁-C₆alkyl, C₄-C₉cycloalkyl, C₄-C₉heterocycloalkyl, aryl, heteroaryl, arylalkyl (e.g., benzyl), and heteroarylalkyl (e.g., pyridylmethyl);
p is 0-3, and
q is 1-5 and r is 0 or
q is 0 and r is 1-5,
or a pharmaceutically acceptable salt thereof. The other variable substituents are as defined above.

Especially useful compounds of formula Id are those wherein R₂ is H, or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

The present invention further relates to compounds of the formula Ie 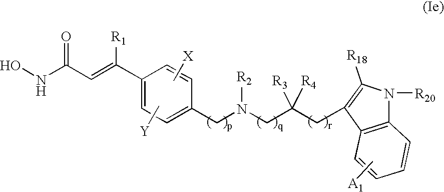
or a pharmaceutically acceptable salt thereof. The variable substituents are as defined above.

Especially useful compounds of formula Ie are those wherein R₁₈ is H, fluoro, chloro, bromo, a C₁-C₄alkyl group, a substituted C₁-C₄alkyl group, a C₃-C₇cycloalkyl group, unsubstituted phenyl, phenyl substituted in the para position, or a heteroaryl (e.g., pyridyl) ring.

Another group of useful compounds of formula Ie are those wherein R₂ is H, or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—H₂—OH and the sum of q and r is preferably 1.

Another group of useful compounds of formula Ie are those wherein R₁₈ is H, methyl, ethyl, t-butyl, trifluoromethyl, cyclohexyl, phenyl, 4-methoxyphenyl, 4-trifluoromethylphenyl, 2-furanyl, 2-thiophenyl, or 2-, 3- or 4-pyridyl wherein the 2-furanyl, 2-thiophenyl and 2-, 3- or 4-pyridyl substituents are unsubstituted or substituted as described above for heteroaryl rings; R₂ is H, or —(CH₂)ₚCH₂OH, wherein p is 1-3; especially those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

Those compounds of formula Ie wherein R₂₀ is H or C₁-C₆alkyl, especially H, are important members of each of the subgenuses of compounds of formula Ie described above.

N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide and N-hydroxy-3-[4-[[[2-(2-methyl-1H-indol-3-yl)-ethyl]-amino]methyl]phenyl]-2E-2-propenamide, or a pharmaceutically acceptable salt thereof, are important compounds of formula Ie.

The present invention further relates to the compounds of the formula If 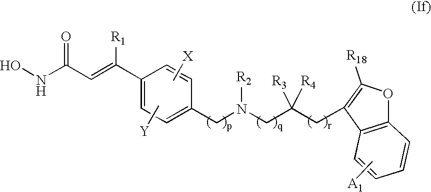
or a pharmaceutically acceptable salt thereof. The variable substituents are as defined above.

Useful compounds of formula If are those wherein R₂ is H, or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

N-hydroxy-3-[4-[[[2-(benzofur-3-yl)-ethyl]-amino]methyl]phenyl]-2E-2-propenamide, or a pharmaceutically acceptable salt thereof, is an important compound of formula If.

The compounds described above are often used in the form of a pharmaceutically acceptable salt. Pharmaceutically acceptable salts include, when appropriate, pharmaceutically acceptable base addition salts and acid addition salts, for example, metal salts, such as alkali and alkaline earth metal salts, ammonium salts, organic amine addition salts, and amino acid addition salts, and sulfonate salts. Acid addition salts include inorganic acid addition salts such as hydrochloride, sulfate and phosphate, and organic acid addition salts such as alkyl sulfonate, arylsulfonate, acetate, maleate, fumarate, tartrate, citrate and lactate. Examples of metal salts are alkali metal salts, such as lithium salt, sodium salt and potassium salt, alkaline earth metal salts such as magnesium salt and calcium salt, aluminum salt, and zinc salt Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylate and benzene sulfonic acid salts.

As is evident to those skilled in the art, the many of the deacetylase inhibitor compounds of the present invention contain asymmetric carbon atoms. It should be understood, therefore, that the individual stereoisomers are contemplated as being included within the scope of this invention.

HDAI compounds within the scope of formula (I) and their synthesis, are disclosed in WO 02/22577 published Mar. 21, 2002 which is incorporated herein by reference in its entirety.

In other embodiments of the present invention the HDAI compound may be selected from any compound that inhibits histone deacetylase such as compounds selected from trapoxin and other tetrapeptides e.g. chlamydocin and HC Toxin; trichostatin and its analogues; apicidin; suberoylanilide hydroxamic acid (SAHA); oxamflatin; MS-275; pyroxamide; valproic acid; FR901228; CI-994; phenylbutyrate; sodium butyrate; 3-(4-aroyl-1H-2pyrrolyl-N-hydroxy-propenamides as disclosed in J. Med. Chem. 45(9):1778-84 (Apr. 25, 2002); ADHA compound 8; -(−)Depudecin; Scriptaid; and Sirtinol.

The HDAC inhibitor compound can be administered as the sole active ingredient or in combination with a second pharmacologically active agent, e.g., together with other immunosuppressive agents, immunomodulating agents, steroids, NSAIDS, or mixtures thereof.

Specific examples of a second pharmacologically active agent include steroids (e.g., methyl prednisolone acetate); immunomodulators (e.g, the sphingosine 1-phosphate receptor agonist FTY-720); NSAIDs; and other known immunosuppressants, such as azathioprine, 15-deoxyspergualin, cyclosporine, mizoribine, mycophenolate mofetil, mycophenolic acid or a salt thereof, brequinar sodium, leflunomide, FK-506 or FK-778. For anti-inflammatory applications, the HDAI compound can be administered with anti-inflammatory agents e.g., corticosteroids such as prednisolone, methylprednisolone and dexamethasone. Dosages of these active agents will vary depending upon the condition and individual to be treated.

Further examples of second pharmacologically active agents include a sphingosine 1-phosphate receptor agonist, e.g. FTY-720 or an analog thereof, e.g. as disclosed in WO 94/08943 which published Apr. 28, 1994, EP 1002792A1, EP0778,263A1, WO02/18395, WO02/076995, WO02/06268, JP-14316985, WO03/29184, WO03/29205, WO03/062252, WO03/062248 or WO03/061567, mTOR inhibitors, e.g. rapamycin, 40-O-(2-hydroxyethyl)-rapamycin and compounds disclosed in WO 94/090101 which published Apr. 28, 1994, calcineurin inhibitors, cyclosporine, CCI779, ABT578, a rapalog or AP23573, AP23464, AP23675 or AP23841; TAFA93, biolimus-7, biolimus-9, an ascomycin having immunosuppressive properties, e.g. ABT-281, ASM981, etc.; cyclophosphamide; methotrexate; a somatostatin analogue like octreotide, lanreotide, vapreotide or SOM230; a deoxyspergualine compound or derivative or analog thereof, e.g. 15-DSG, monoclonal antibodies to leukocyte receptors, e.g., MHC, CD2, CD3, CD4, CD7, CD8, CD11a/CD18, CD25, CD27, CD28, CD40. CD45, CD58, CD80, CD86, CD134, CD137, ICOS, CD150 (SLAM), CD152, OX40, 4-1BB or to their ligands, e.g. CD154, or antagonists thereof; other immunomodulatory compounds, e.g. a recombinant binding molecule having at least a portion of the extracellular domain of CTLA4 or a mutant thereof, e.g. an at least extracellular portion of CTLA4-or a mutant thereof joined to a non-CTLA4 protein sequence, e.g. CTLA4Ig (for ex. designated ATCC 68629) or a homologue or a mutant thereof, e.g. LEA29Y; adhesion molecule inhibitors, e.g. LFA-1 antagonists, ICAM-1 or -3 antagonists, anti-LFA-1 or anti-ICAM antibodies, VCAM-4 antagonists or VLA-4 antagonists; or anti-chemokine antibodies or anti-chemokine receptor antibodies or low molecular weight chemokine receptor antagonists, e.g. anti MCP-1 antibodies. Preferably, the agent effective in preventing, delaying or treating transplant rejection is a calcineurin inhibitor, most preferably cyclosporin A, FK506 or FK778.

The structure of the active agents cited and identified by code numbers, generic or trademark names may be taken from the actual edition of the standard compendium “The Merck Index” or from databases, e.g. Patents International (e.g. IMS World Publications). The corresponding content thereof is hereby incorporated by reference. Any person skilled in the art is fully enable, based on these references, to manufacture and test the pharmaceutical indications and properties in standard test models, both in vitro and in vivo.

The term “coadministration” or “combined administration” or the like as utilized herein are meant to encompass administration of the selected therapeutic agents to a single patient, and are intended to include treatment regimens in which the agents are not necessarily administered by the same route or administration or at the same time.

The invention further also relates to a method for the treatment, prevention or suppression of an immune disorder (especially autoimmune disease or inflammatory disease), immune response or immune mediated response, or for the prevention or treatment of acute or chronic transplant rejection in a recipient patient of an organ, tissue or cell transplant which comprises treating the mammal with pharmaceutically effective amounts of a histone deacetylase inhibitor, especially selected from those mentioned herein, most especially from those mentioned as being preferred.

In another embodiment the present invention relates to a pharmaceutical composition which comprises a histone deacetylase inhibitor, especially selected from those mentioned herein, most especially from those mentioned as being preferred, or a pharmaceutically acceptable salt thereof or a pharmaceutically acceptable prodrug thereof, optionally together with at least one pharmaceutically acceptable carrier for use in the treatment, prevention or suppression of an immune disorder, immune response or immune mediated response, especially where the immune disorder is selected from autoimmune disease or inflammatory disease, or for the prevention or treatment of acute or chronic transplant rejection in a recipient patient of an organ, tissue or cell transplant.

In a further embodiment the invention relates to the use of a histone deacetylase inhibitor, especially selected from those mentioned herein, most especially from those mentioned as being preferred, for the preparation of a pharmaceutical composition for use in the treatment, prevention or suppression of an immune disorder, immune response or immune mediated response, or for the prevention or treatment of acute or chronic transplant rejection in a recipient patient of an organ, tissue or cell transplant.

In another embodiment the present invention relates to a commercial package or product comprising a histone deacetylase inhibitor, especially selected from those mentioned herein, most especially from those mentioned as being preferred, or a pharmaceutically acceptable salt thereof or a pharmaceutically acceptable prodrug thereof.

Cellular Assay: Allogeneic Mixed Lymphocyte Reaction (MLR):

Agents of the invention exhibit T cell inhibiting activity. More particular the agents of the invention prevent T cell activation and/or proliferation in e.g. aqueous solution, e.g. as demonstrated in accordance with the following test method. The two-way MLR is performed according to standard procedures (J. Immunol. Methods, 1973, 2, 279 and Meo T. et al., Immunological Methods, New York, Academic Press, 1979, 227-39). Briefly, spleen cells from CBA and BALB/c mice (1.6×10⁵ cells from each strain per well in flat bottom tissue culture microtiter plates, 3.2×10⁵ in total) are incubated in RPMI medium containing 10% FCS, 100 U/ml penicillin, 100 μg/ml streptomycin (Gibco BRL, Basel, Switzerland), 50 μM 2-mercaptoethanol (Fluka, Buchs, Switzerland) and serially diluted compounds. Seven three-fold dilution steps in duplicates per test compound are performed. After four days of incubation 1 μCi ³H-thymidine is added. Cells are harvested after an additional five-hour incubation period, and incorporated ³H-thymidine is determined according to standard procedures. Background values (low control) of the MLR are the proliferation of BALB/c cells alone. Low controls are subtracted from all values. High controls without any sample are taken as 100% proliferation. Percent inhibition by the samples is calculated, and the concentrations required for 50% inhibition (IC₅₀ values) are determined. In this assay, the agents of the invention have IC₅₀ values in the range of 1 nM to 10 μM, preferably from 1 nM to 500 nM. A compound of formula (I), e.g. N-hydroxy-3-[4-[[[2-(2-methyl-1H-indol-3-yl)ethyl]-amino]methyl]phenyl]-2E-2-propenamide shows an IC₅₀ value of 9 nM.

Combinations

Thus, in another aspect, the present invention relates to a combination which comprises (a) a histone deacetylase inhibitor, especially selected from those mentioned herein, most especially from those mentioned as being preferred, and (b) a second pharmacologically active agent, especially selected from those mentioned herein, most especially from those mentioned as being preferred, in which (a) and (b) are present in each case in free form or in the form of a pharmaceutically acceptable salt or a pharmaceutically acceptable prodrug thereof, for simultaneous, concurrent, separate or sequential use.

In another embodiment the invention relates a combination which comprises (a) a histone deacetylase inhibitor, especially selected from those mentioned herein, most especially from those mentioned as being preferred, and (b) a second pharmacologically active agent, especially selected from those mentioned herein, most especially from those mentioned as being preferred, in which (a) and (b) are present in each case in free form or in the form of a pharmaceutically acceptable salt or a pharmaceutically acceptable prodrug thereof, for use in the treatment, prevention or suppression of an immune disorder, immune response or immune mediated response, or for the prevention or treatment of acute or chronic transplant rejection in a recipient patient of an organ, tissue or cell transplant.

The present invention also relates to the use of a histone deacetylase inhibitor, especially selected from those mentioned herein, most especially from those mentioned as being preferred, or a pharmaceutically acceptable salt thereof or a pharmaceutically acceptable prodrug thereof, for the preparation of a medicament or pharmaceutical composition, for use in combination with a second pharmacologically active agent, especially selected from those mentioned herein, most especially from those mentioned as being preferred, or a pharmaceutically acceptable salt thereof or a pharmaceutically acceptable prodrug thereof, for use in the treatment, prevention or suppression of an immune disorder, immune response or immune mediated response, or for the prevention or treatment of acute or chronic transplant rejection in a recipient patient of an organ, tissue or cell transplant.

In another embodiment the present invention relates to a pharmaceutical composition which comprises (a) a histone deacetylase inhibitor, especially selected from those mentioned herein, most especially from those mentioned as being preferred, or a pharmaceutically acceptable salt thereof or a pharmaceutically acceptable prodrug thereof, and (b) a second pharmacologically active agent, especially selected from those mentioned herein, most especially from those mentioned as being preferred, or a pharmaceutically acceptable salt thereof or a pharmaceutically acceptable prodrug thereof, together with at least one pharmaceutically acceptable carrier.

In another embodiment the present invention relates to a commercial package or product comprising a histone deacetylase inhibitor, especially selected from those mentioned herein, most especially from those mentioned as being preferred, or a pharmaceutically acceptable salt thereof or a pharmaceutically acceptable prodrug thereof, or a commercial package or product comprising a histone deacetylase inhibitor, especially selected from those mentioned herein, most especially from those mentioned as being preferred, or a pharmaceutically acceptable salt thereof or a pharmaceutically acceptable prodrug thereof, together with instructions for use in combination with a second pharmacologically active agent, especially selected from those mentioned herein, most especially from those mentioned as being preferred, or a pharmaceutically acceptable salt thereof or a pharmaceutically acceptable prodrug thereof, for the treatment, prevention or suppression of an immune disorder, immune response or immune mediated response, or for the prevention or treatment of acute or chronic transplant rejection in a recipient patient of an organ, tissue or cell transplant.

The invention also relates to a commercial package or product comprising a combination which comprises (a) a histone deacetylase inhibitor, especially selected from those mentioned herein, or a pharmaceutically acceptable salt thereof or a pharmaceutically acceptable prodrug thereof, and (b) a second pharmacologically active agent, especially selected from those mentioned herein, in which (a) and (b) are present in each case in free form or in the form of a pharmaceutically acceptable salt or a pharmaceutically acceptable prodrug thereof, together with instructions for simultaneous, concurrent, separate or sequential use thereof for the treatment, prevention or suppression of an immune disorder, immune response or immune mediated response, or for the prevention or treatment of acute or chronic transplant rejection in a recipient patient of an organ, tissue or cell transplant.

The present invention further relates to “a combined preparation”, which, as used herein, defines especially a “kit of parts” in the sense that the combination partners (a) and (b) as defined above can be dosed independently or by use of different fixed combinations with distinguished amounts of the combination partners (a) and (b), i.e., simultaneously or at different time points. The parts of the kit of parts can then, e.g., be administered simultaneously or chronologically staggered, that is at different time points and with equal or different time intervals for any part of the kit of parts. The ratio of the total amounts of the combination partner (a) to the combination partner (b) to be administered in the combined preparation can be varied, e.g. in order to cope with the needs of a patient sub-population to be treated or the needs of the single patient based on the severity of any side-effects that the patient experiences.

The invention further also relates to a method for the treatment, prevention or suppression of an immune disorder, immune response or immune mediated response, or for the prevention or treatment of acute or chronic transplant rejection in a recipient patient of an organ, tissue or cell transplant which comprises treating the mammal with pharmaceutically effective amounts of a combination which comprises (a) a histone deacetylase inhibitor, especially selected from those mentioned herein, most especially from those mentioned as being preferred, and (b) a second pharmacologically active agent, especially selected from those mentioned herein, most especially from those mentioned as being preferred, in which (a) and (b) are present in each case in free form or in the form of a pharmaceutically acceptable salt or a pharmaceutically acceptable prodrug thereof.

Preferred combinations for any of the above mentioned embodiments are those where the HDAI is selected from the group consisting of N-hydroxy-3-[4-[[(2-hydroxyethyl)[2-(1H-indol-3-yl)ethyl]-amino]methyl]phenyl]-2E-2-propenamide; N-hydroxy-3-[4-[[[2-(1H-indol-3-yl)ethyl]-amino]methyl]phenyl]-2E-2-propenamide; N-hydroxy-3-[4-[[[2-(2-methyl-1H-indol-3-yl)-ethyl]-amino]methyl]phenyl]-2E-2-propenamide, SAHA and pharmaceutically acceptable salts thereof, and the second pharmacologically active agent is selected from FTY720 or 40-O-(2-hydroxyethyl)-rapamycin.

According to the present invention, a patient is treated simultaneously, concurrently, separately or sequentially with pharmaceutically effective amounts of a combination of an HDAI and a second pharmacologically active agent in order to treat, prevent or suppress an immune disorder, immune response or immune mediated response, or to prevent or treat an acute or chronic transplant rejection in a recipient patient of an organ, tissue or cell transplant, each according to a dosage regimen that is appropriate for the individual agent. For example, the second pharmacologically active agent may be administered once or more daily and the HDAI may be administered once daily, on alternate days or on some other schedule—as is appropriate for the HDAI agent when used without the pharmacologically active agent. One of skill in the art has the ability to determine appropriate pharmaceutically effective amounts of the combination components.

Co-administration of an HDA inhibitor and an second pharmaceutically active agent may result in a synergistic-effect which effect is greater than the sum of the effect achieved for the either compound separately. Specifically, a synergistic effect is observed with an HDAI is co-administered with an immunomodulator such as sphingosine 1-phosphate receptor agonist. A synergistic effect is also seen when an HDAI is co-administered with an mTOR inhibitor, e.g. 40-O-(2-hydroxyethyl)-rapamycin. Specifically a synergistic effect is seen when N-hydroxy-3-[4-[[[2-(2-methyl-1H-indol-3-yl)-ethyl]-amino]methyl]phenyl]-2E-2-propenamide is co-administered with a sphingosine 1-phosphate receptor agonist. Further a synergistic effect is seen when N-hydroxy-3-[4-[[[2-(2-methyl-1H-indol-3-yl)-ethyl]-amino]methyl]phenyl]-2E-2-propenamide is co-administered with a mTOR inhibitor, e.g. 40-O-(2-hydroxyethyl)-rapamycin.

Pharmaceutical compositions of the present invention comprise an effective amount of active compound(s) in conjunction or admixture with excipients or carriers suitable for either enteral or parenteral application. Tablets and gelatin capsules may comprise the active compound(s) together with diluents; lubricants, binders, disintegrants; and/or absorbents, colorants, flavors and sweeteners.

Injectable compositions are preferably aqueous isotonic solutions or suspensions, and suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, the compositions may also contain other therapeutically valuable substances.

Suitable formulations also include formulations for parenteral administration such as aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The compositions may be sterilized and/or contain adjuvants, such as preserving, stabilizing, wetting or emulsifying agents, solution promoters, salts for regulating the osmotic pressure and/or buffers. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

Additional routes of administration include topical applications, including transdermal, ocular, buccal, intranasal, inhalation, intravaginal, rectal, and intracistemal.

The compositions are prepared according to conventional mixing, granulating or coating methods, respectively, and contain preferably about 1 to 50% of the active ingredient. The pharmaceutically acceptable carriers or excipients are selected on the basis of the chosen route of administration and standard pharmaceutical practice.

Suitable dosages will be dependent on the age, health and weight of the recipient, the extent of the disease, kind of concurrent treatment, if any, frequency of treatment and the nature of the effect desired. The same dosage forms can generally be used when the compounds of this invention are administered stepwise or in conjunction with another therapeutic agent.

In general, oral dosages for the HDAI compound are on the order of from 0.05 to 5 or up to 10 mg/kg/day, e.g. on the order of from 0.1 to 2 or up to 7.5 mg/kg/day administered once or, in divided doses 2 to 4 times per day, or on administration parenterally, e.g. intravenously, for example by i.v. drip or infusion, at dosages on the order of from 0.01 to 2.5 up to 5 mg/kg/day, e.g. on the order of from 0.05 or 0.1 up to 1.0 mg/kg/day. Suitable daily dosages for patients are thus on the order of 500 mg p.o., e.g. on the order of from 5 to 100 mg p.o., or on the order of from 0.5 to 125 up to 250 mg i.v., e.g. on the order of from 2.5 to 50 mg i.v.

The S1P receptor agonist, e.g. FTY 720, may be administered by any conventional route, in particular enterally, e.g. orally, e.g. in the form of tablets, capsules, drink solutions or parenterally, e.g. in the form of injectable solutions or suspensions. Suitable unit dosage forms for oral administration comprise from ca. 0.02 to 50 mg active ingredient, usually 0.1 to 30 mg, e.g. S1P receptor agonist, together with one or more pharmaceutically acceptable diluents or carriers therefor.

Dosages required in practicing the method of the present invention when the second pharmaceutically active agent is an mTOR inhibitor, e.g. 40-O-(2-hydroxyethyl)-rapamycin, will vary depending upon, for example, the compound used, the host, the mode of administration and the severity of the condition to be treated. A preferred daily dosage range is about from 0.1 to 25 mg as a single dose or in divided doses. Suitable daily dosages for patients are on the order of from e.g. 0.1 to 25 mg p.o.

Cyclosporine can be administered by conventional means, preferably by oral doses ranging from 1-250 mg, preferably 25-100 mg or injectable solutions in the range of 25-100 mg/ml.

Due to the synergistic aspect of combinations of the present invention, it may be possible to use smaller dosage amounts of the active ingredients and still obtain positive effective results.

Dosage forms of the present invention will include an HDAI compound, optional second active compound and pharmaceutically acceptable excipients, and when the second active compound is present as separate dosage forms or as a dosage form which is a fixed combination. “An effective amount” is the dosage of compound required to achieve the desired therapeutic and/or prophylactic effect; for example, the dosage of the compound which results in suppression of an immune response in the subject, or which results in suppression of an organ transplant rejection in the subject. An effective amount of the compound can be administered by an appropriate route in a single dose or in multiple doses. As used herein, a “subject” refers to an animal such as a mammal, human or animal subject in need of veterinary treatment.

The HDAI compounds can be administered for the treatment of autoimmune diseases, the prevention of rejection of foreign organ transplants and/or related afflictions, diseases and illnesses.

In a further embodiment, the present invention is directed to a method of preventing or treating manifestations of chronic rejection in a recipient of organ or tissue transplant, e.g. heart, lung, combined heart-lung, trachea, liver, bowel, kidney or pancreatic transplants, comprising administering a therapeutically effective amount of an HDAI compound of formula I, in free form or in pharmaceutically acceptable salt form.

The HDAI compounds may be administered to treat, prevention or suppress the immune response in subjects having autoimmune disease, inflammatory disease, or graft-versus-host disease, as well as to subjects having undergone an allogeneic transplant or xenogeneic transplant. Further methods include administration of an HDAI compound for inhibiting the proliferation of lymphocytes, and/or for enhancing graft survival following transplant by administration previous to, concurrently with, or subsequent to a transplant procedure (as used herein, transplant includes allogeneic and xenogeneic transplant).

The present invention is related to the use of an HDAI compound in a subject for the treatment and/or prevention of immune response or immune-mediated responses and diseases, such as the prevention or treatment of rejection following transplantation of synthetic or organic grafting materials, cells, organs or tissue to replace all or part of the function of tissues, such as heart, kidney, liver, bone marrow, skin, cornea, vessels, lung, pancreas, intestine, limb, muscle, nerve tissue, duodenum, small-bowel, pancreatic-islet-cell, including xeno-transplants, etc.; to treat or prevent graft-versus-host disease. The HDAI compounds are also useful for treating and preventing autoimmune diseases and inflammatory conditions, in particular inflammatory conditions with an etiology including an autoimmune component such as arthritis (for example rheumatoid arthritis, arthritis chronica progrediente and arthritis deformans) and rheumatic diseases. Specific autoimmune diseases for which the compounds of the invention may be employed include, autoimmune hematological disorders (including e.g. hemolytic anaemia, aplastic anaemia, pure Ted cell anaemia and idiopathic thrombocytopenia), systemic lupus erythematosus, thyroiditis, Hashimoto's thyroiditis, polychondritis, sclerodoma, Wegener granulamatosis, dermatomyositis, chronic active hepatitis, myasthenia gravis, psoriasis, atopic dermatitis, vasculitis, Steven-Johnson syndrome, idiopathic sprue, autoimmune inflammatory bowel disease (including e.g. ulcerative colitis and Crohn's disease) endocrine ophthalmopathy, Graves disease, sarcoidosis, multiple sclerosis, primary billiary cirrhosis, juvenile diabetes (diabetes mellitus type I), diabetes type II and the disorders associated therewith, uveitis (anterior and posterior), keratoconjunctivitis sicca and vernal keratoconjunctivitis, interstitial lung fibrosis, psoriatic arthritis, glomerulonephritis (with and without nephrotic syndrome, e.g. including idiopathic nephrotic syndrome or minimal change nephropathy) and juvenile dermatomyositis.

The HDAI compounds may also be administered to treat or prevent auto-antibody mediated diseases, aplastic anemia, Evan's syndrome, autoimmune hemolytic anemia, and the like; and further to treat infectious diseases causing aberrant immune response and/or activation, such as traumatic or pathogen induced immune disregulation, including for example, that which are caused by hepatitis B and C infections, staphylococcus aureus infection, viral encephalitis, sepsis, parasitic diseases wherein damage is induced by an inflammatory response (e.g., leprosy); and to prevent or treat circulatory diseases, such as arteriosclerosis, atherosclerosis, vasculitis, polyarteritis nodosa and myocarditis. In addition the present invention may be used to prevent/suppress an immune response associated with a gene therapy treatment, such as the introduction of foreign genes into autologous cells and expression of the encoded product.

As used herein, “an immune response” refers to the body's reaction to foreign or self antigens so that they are neutralized and/or eliminated. The term “tolerance,” as used herein, refers to a state of non-responsiveness of the immune system toward an antigen that it has the ability to react against.

Accordingly, an embodiment of the invention is a method for the treatment of autoimmune diseases by the administration of an HDAI compound. While, another embodiment of the invention is a method for the prevention or treatment of rejection of foreign organ transplants comprising administering to a patient in need of such therapy a therapeutically effective amount of an HDAI compound.

As used herein, the term “graft” refers to organs and/or tissues which can be obtained from a first mammal (or donor) and transplanted into a second mammal (or recipient), preferably a human. The term “graft” encompasses, for example, skin, eye or portions of the eye (e.g., cornea, retina, lens), muscle, bone marrow or cellular components of the bone marrow (e.g., stem cells, progenitor cells), heart, lung, heart-lung, liver, kidney, pancreas (e.g., islet cells, β-cells), parathyroid, bowel (e.g., colon, small intestine, duodenum), neuronal tissue, bone and vasculature (e.g., artery, vein). A graft can be obtained from suitable mammal (e.g., human or pig), or under certain circumstances a graft can be produced in vitro by culturing cells, for example embryonal, skin or blood cells and bone marrow cells. A graft is preferably obtained from human.

The following Example illustrates the invention described above; it is not, however, intended to limit the scope of the invention in any way. The beneficial effects of the combination of the invention can also be determined by other test models known as such to the person skilled in the pertinent art.

The following examples are provided here for purposes of illustration and not intended to limit the scope of the present invention.

### EXAMPLE I

Acute Rejection:

Balb/c (H-2^{d}) mice are used as donor animals, and e.g. C57BL/6J (H-2^{b}) mice as recipients. The heart is removed from the donors according to known procedures and stored in cold saline (4° C.). The recipient animals are anaesthetised with isofluorane. Infrarenal abdominal aorta and inferior vena cava are exposed. Blood vessels are dissected free from the fascia for a length of 3-5 mm, ligating and dividing any small branches. Vessels are occluded, first proximally and then distally. An arteriotomy and venotomy is performed, and lumens are flushed with heparinised physiological saline. End-to-side aortic anastomosis and then end-to-side anastomosis of the donor right pulmonary to recipient inferior vena cava are performed. The distal ligature is removed, then the proximal ligature. The suture lines are checked for leakage. Then the graft is tethered retroabdominally. The abdomen is flooded with warm saline (37° C.) and the wound is closed. Graft function is monitored daily by palpation of the abdomen. Rejection is concluded when the graft stops beating. Animals are treated with a 100 μL subcutaneous injection of saline or a solution of a HDAI, e.g. the compound of formula (I), e.g. N-hydroxy-3-[4-[[[2-(2-methyl-1H-indol-3-yl)-ethyl]-amino]methyl]phenyl]-2E-2-propenamide in lactate buffer pH 5.5 according to the dosing schedule shown in Table 1 below:

TABLE 2

| Experiment | Dose | Graft Survival (Days) |
|---|---|---|
| A | Saline | 7, 7, 7, 7 |
| B | 2.5 mg/kg once daily | 14, 15, 13, 16, 19 |
| C | 5 mg/kg once daily | 14, 20, 22, 23 |
| | (day 0-14 only) | |
| D | 5 mg/kg twice a day | 24, 34 |
| | (day 0-12 only) | |

As can be seen by the above results shown in Table 1, administration of N-hydroxy-3-[4-[[[2-(2-methyl-1H-indol-3-yl)-ethyl]-amino]methyl]phenyl]-2E-2-propenamide increases graft survival by 2-5 times the number of days.

### EXAMPLE 2

Rat Heart Transplantation

The strain combination used is Male Lewis (RT1 haplotype) and BN (RT1 haplotype). The animals are anaesthetized using inhalation isofluorane. Following heparinization of the donor rat through the abdominal inferior vena cava with simultaneous exsanguination via the aorta, the chest is opened and the heart rapidly cooled. The aorta is ligated and divided distal to the first branch and the brachiocephalic trunk is divided at the first bifurcation. The left pulmonary artery is ligated and divided and the right side divided but left open. All other vessels are dissected free, ligated and divided and the donor heart is removed into iced saline.

The recipient is prepared by dissection and cross-clamping of the infra-renal abdominal aorta and vena cava. The graft is implanted with end-to-side anastomoses, using 10/0 monofilament suture, between the donor brachiocephalic trunk and the recipient aorta and the donor right pulmonary artery to the recipient vena cava. The clamps are removed, the graft tethered retroabdominally, the abdominal contents washed with warm saline and the animal is closed and allowed to recover under a heating lamp. Animals are treated with saline (control); an HDAI compound e.g. a compound of formula (I), e.g. N-hydroxy-3-[4-[[[2-(2-methyl-1H-indol-3-yl)-ethyl]-amino]methyl]phenyl]-2E-2-propenamide (“compound P1”); 40-O-(2-hydroxyethyl)-rapamycin (“compound P2”) prepared according to the methods described in WO 94/09010; FTY720 (“compound P3”) prepared according to Example 28 of WO 94/08943, or combinations of the each as indicated. Graft survival is monitored by daily palpation of the beating donor heart through the abdominal wall. Rejection is considered to be complete when heart beat stops. At that point the animal is euthanized and the transplanted heart is fixed for histology. Table 2 below summarizes the survival and histology data. The scale for grading for acute rejection is 1=slight; 2=moderate; 3=marked; 4=severe.

TABLE 2

| | Dosis | Dosis | Survival (days) | Median | Grading for acute cardiac |
|---|---|---|---|---|---|
| | (mg/kg/d) | (mg/kg/d) | for each | survival time | rejection for each |
| Compound | s.c. | p.o. | individual graft | (MST) | individual graft |
| Control | | 0 | 6, 7, 7, 8 | 7 | 4, 4, 4, 4 |
| Compound P1 | 0.3 | | 6, 6, 6, 7 | 6 | 3, 3, 4, 4 |
| Compound P1 | 1.0 | | >28, >28, >28 | >28 | 1, 2, 2 |
| Compound P2 | | 0.3 | 7, 7, 7, 7, 8, 8 | 7 | 4, 4, 4, 4, 4, 4, |
| Compound P1 + | 0.3 | 0.3 | >28, >28, >28, | >28 | 1, 2, 2, 3 |
| Compound P2 | | | >28 | | |
| Compound P3 | | 0.1 | 7, 7, 7, 8, 8, 8 | 7.5 | 4, 4, 4, 4, 4, 3 |
| Compound P1 + | 0.3 | 0.1 | >28, >28, >28 | >28 | 1, 1, 1, 1 |
| Compound P3 | | | | | |

### EXAMPLE 3

Example 1 can be repeated using N-Hydroxy-3-[4-[[(2-hydroxyethyl)[2-(1H-indol-3-yl)-ethyl]-amino]methyl]phenyl]-2E-2-propenamide as the HDAI compound, and following the same process as shown in Example 1.

### EXAMPLE 4

Example 1 can be repeated using suberoylanilide hydroxamic acid (SAHA), as the HDAI compound, and following the same process as shown in Example 1.

### EXAMPLE 5

Example 2 can be repeated using N-Hydroxy-3-[4-[[(2-hydroxyethyl)[2-(1H-indol-3-yl)-ethyl]-amino]methyl]phenyl]-2E-2-propenamide, as the HDAI compound, and following the same process as shown in Example 2.

### EXAMPLE 6

Example 2 can be repeated using suberoylanilide hydroxamic acid (SAHA), instead of the compound P3 and following the same process as shown in Example 2.

1-15. (canceled)

16. A method of treating, preventing or suppressing an immune disorder, immune response or immune mediated response of an animal comprising administering to said animal an effective amount of an histone deacetylase inhibitor compound of formula I: 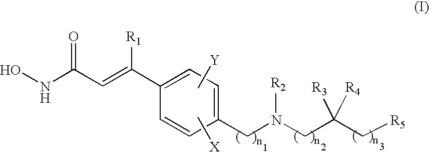
wherein
R₁ is H, halo, or a straight chain C₁-C₆ alkyl;
R₂ is selected from H, C₁-C₁₀ alkyl, C₄-C₉ cycloalkyl, C₄-C₉ heterocycloalkyl, C₄-C₉ heterocycloalkylalkyl, cycloalkylalkyl, aryl, heteroaryl, arylalkyl, heteroarylalkyl, —(CH₂)ₙC(O)R₆, —(CH₂)ₙOC(O)R₆, amino acyl, HON—C(O)—CH═C(R₁)-aryl-alkyl- and —(CH₂)ₙR₇;
R₃ and R₄ are the same or different and independently H, C₁-C₆ alkyl, acyl or acylamino, or R₃ and R₄ together with the carbon to which they are bound represent C═O, C═S, or C═NR₈, or R₂ together with the nitrogen to which it is bound and R₃ together with the carbon to which it is bound can form a C₄-C₉ heterocycloalkyl, a heteroaryl, a polyheteroaryl, a non-aromatic polyheterocycle, or a mixed aryl and non-aryl polyheterocycle ring;
R₅ is selected from H, C₁-C₆ alkyl, C₄-C₉ cycloalkyl, C₄-C₉ heterocycloalkyl, acyl, aryl, heteroaryl, arylalkyl, heteroarylalkyl, aromatic polycycle, non-aromatic polycycle, mixed aryl and non-aryl polycycle, polyheteroaryl, non-aromatic polyheterocycle, and mixed aryl and non-aryl polyheterocycle;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H, halo, C₁-C₄ alkyl, NO₂, C(O)R₁, OR₉, SR₉, CN, and NR₁₀R₁₁;
R₆ is selected from H, C₁-C₆ alkyl, C₄-C₉ cycloalkyl, C₄-C₉ heterocycloalkyl, cycloalkylalkyl, aryl, heteroaryl, arylalkyl, heteroarylalkyl, OR₁₂, and NR₁₃R₁₄;
R₇ is selected from OR₁₅, SR₁₅, S(O)R₁₆, SO₂R₁₇, NR₁₃R₁₄, and NR₁₂SO₂R₆;
R₈ is selected from H, OR₁₅, NR₁₃R₁₄, C₁-C₆ alkyl, C₄-C₉ cycloalkyl, C₄-C₉ heterocycloalkyl, aryl, heteroaryl, arylalkyl, and heteroarylalkyl;
R₉ is selected from C₁-C₄ alkyl and C(O)-alkyl;
R₁₀ and R₁₁ are the same or different and independently selected from H, C₁-C₄ alkyl, and —C(O)-alkyl;
R₁₂ is selected from H, C₁-C₆ alkyl, C₄-C₉ cycloalkyl, C₄-C₉ heterocycloalkyl, C₄-C₉ heterocycloalkylalkyl, aryl, mixed aryl and non-aryl polycycle, heteroaryl, arylalkyl, and heteroarylalkyl;
R₁₃ and R₁₄ are the same or different and independently selected from H, C₁-C₆ alkyl, C₄-C₉ cycloalkyl, C₄-C₉ heterocycloalkyl, aryl, heteroaryl, arylalkyl, heteroarylalkyl, amino acyl, or R₁₃ and R₁₄ together with the nitrogen to which they are bound are C₄-C₉ heterocycloalkyl, heteroaryl, polyheteroaryl, non-aromatic polyheterocycle or mixed aryl and non-aryl polyheterocycle;
R₁₅ is selected from H, C₁-C₆ alkyl, C₄-C₉ cycloalkyl, C₄-C₉ heterocycloalkyl, aryl, heteroaryl, arylalkyl, heteroarylalkyl and (CH₂)ₘZR₁₂;
R₁₆ is selected from C₁-C₆ alkyl, C₄-C₉ cycloalkyl, C₄-C₉ heterocycloalkyl, aryl, heteroaryl, polyheteroaryl, arylalkyl, heteroarylalkyl and (CH₂)ₘZR₁₂;
R₁₇ is selected from C₁-C₆ alkyl, C₄-C₉ cycloalkyl, C₄-C₉ heterocycloalkyl, aryl, aromatic polycycle, heteroaryl, arylalkyl, heteroarylalkyl, polyheteroaryl and NR₁₃R₁₄;
m is an integer selected from 0 to 6; and
Z is selected from O, NR₁₃, S and S(O);
or a pharmaceutically acceptable salt thereof.

17. A method for preventing or treating acute or chronic transplant rejection in a recipient patient of organ or tissue or cell transplant comprising the step of administering to said patient a therapeutically effective amount of a compound of formula (I) according to claim 16.

18. A method according to claim 16 further comprising a second pharmacologically active agent.

19. A method according to claim 18 wherein the second pharmacologically active agent is selected from immunosuppressive agents, immunomodulating agents, antibiotics, antiviral agents, steroids, NSAIDS or mixtures thereof.

20. A method for enhancing graft survival following transplant, comprising administering to an animal previous to, concurrently with, or subsequent to a transplant procedure an effective amount of an histone deacetylase inhibitor compound of formula I according to claim 16.

21. A combination for use in the treatment, prevention or suppression of an immune disorder, immune response or immune mediated response, or for the prevention or treatment of acute or chronic transplant rejection in a recipient patient of an organ, tissue or cell transplant,
which comprises (a) a histone deacetylase inhibitor and (b) a second pharmacologically active agent in which (a) and (b) are present in each case in free form or in the form of a pharmaceutically acceptable salt or a pharmaceutically acceptable prodrug thereof, for simultaneous, concurrent, separate or sequential use.

22. The combination according to claim 21 wherein the histone deacetylase inhibitor is a compound of formula (I): 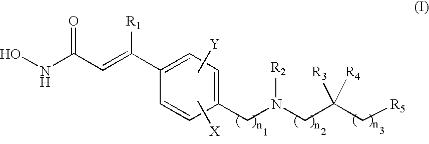
wherein
R₁ is H, halo, or a straight chain C₁-C₆ alkyl;
R₂ is selected from H, C₁-C₁₀ alkyl, C₄-C₉ cycloalkyl, C₄-C₉ heterocycloalkyl, C₄-C₉ heterocycloalkylalkyl, cycloalkylalkyl, aryl, heteroaryl, arylalkyl, heteroarylalkyl, —(CH₂)ₙC(O)R₆, —(CH₂)ₙOC(O)R₆, amino acyl, HON—C(O)—CH═C(R₁)-aryl-alkyl- and —(CH₂)ₙR₇;
R₃ and R₄ are the same or different and independently H, C₁-C₆ alkyl, acyl or acylamino, or R₃ and R₄ together with the carbon to which they are bound represent C═O, C═S, or C═NR₈, or R₂ together with the nitrogen to which it is bound and R₃ together with the carbon to which it is bound can form a C₄-C₉ heterocycloalkyl, a heteroaryl, a polyheteroaryl, a non-aromatic polyheterocycle, or a mixed aryl and non-aryl polyheterocycle ring;
R₅ is selected from H, C₁-C₆ alkyl, C₄-C₉ cycloalkyl, C₄-C₉ heterocycloalkyl, acyl, aryl, heteroaryl, arylalkyl, heteroarylalkyl, aromatic polycycle, non-aromatic polycycle, mixed aryl and non-aryl polycycle, polyheteroaryl, non-aromatic polyheterocycle, and mixed aryl and non-aryl polyheterocycle;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H, halo, C₁-C₄ alkyl, NO₂, C(O)R₁, OR₉, SR₉, CN, and NR₁₀R₁₁;
R₆ is selected from H, C₁-C₆ alkyl, C₄-C₉ cycloalkyl, C₄-C₉ heterocycloalkyl, cycloalkylalkyl, aryl, heteroaryl, arylalkyl, heteroarylalkyl, OR₁₂, and NR₁₃R₁₄;
R₇ is selected from OR₁₅, SR₁₅, S(O)R₁₆, SO₂R₁₇, NR₁₃R₁₄, and NR₁₂SO₂R₆;
R₈ is selected from H, OR₁₅, NR₁₃R₁₄, C₁-C₆ alkyl, C₄-C₉ cycloalkyl, C₄-C₉ heterocycloalkyl, aryl, heteroaryl, arylalkyl, and heteroarylalkyl;
R₉ is selected from C₁-C₄ alkyl and C(O)-alkyl;
R₁₀ and R₁₁ are the same or different and independently selected from H, C₁-C₄ alkyl, and —C(O)-alkyl;
R₁₂ is selected from H, C₁-C₆ alkyl, C₄-C₉ cycloalkyl, C₄-C₉ heterocycloalkyl, C₄-C₉ heterocycloalkylalkyl, aryl, mixed aryl and non-aryl polycycle, heteroaryl, arylalkyl, and heteroarylalkyl;
R₁₃ and R₁₄ are the same or different and independently selected from H, C₁-C₆ alkyl, C₄-C₉ cycloalkyl, C₄-C₉ heterocycloalkyl, aryl, heteroaryl, arylalkyl, heteroarylalkyl, amino acyl, or R₁₃ and R₁₄ together with the nitrogen to which they are bound are C₄-C₉ heterocycloalkyl, heteroaryl, polyheteroaryl, non-aromatic polyheterocycle or mixed aryl and non-aryl polyheterocycle;
R₁₅ is selected from H, C₁-C₆ alkyl, C₄-C₉ cycloalkyl, C₄-C₉ heterocycloalkyl, aryl, heteroaryl, arylalkyl, heteroarylalkyl and (CH₂)ₘZR₁₂;
R₁₆ is selected from C₁-C₆ alkyl, C₄-C₉ cycloalkyl, C₄-C₉ heterocycloalkyl, aryl, heteroaryl, polyheteroaryl, arylalkyl, heteroarylalkyl and (CH₂)ₘZR₁₂;
R₁₇ is selected from C₁-C₆ alkyl, C₄-C₉ cycloalkyl, C₄-C₉ heterocycloalkyl, aryl, aromatic polycycle, heteroaryl, arylalkyl, heteroarylalkyl, polyheteroaryl and NR₁₃R₁₄;
m is an integer selected from 0 to 6; and
Z is selected from O, NR₁₃, S and S(O);
or a pharmaceutically acceptable salt thereof.

23. The combination of claim 21 wherein the second pharmacologically active agent is selected from immunosuppressive agents, immunomodulating agents, steroids, NSAIDS or mixtures thereof.

24. The combination of claim 21 wherein the second pharmacologically active agent is selected from sphingosine 1-phosphate receptor agonist, e.g. FTY-720 or an analog thereof, mTOR inhibitors, e.g. rapamycin, 40-O-(2-hydroxyethyl)-rapamycin; calcineurin inhibitors, cyclosporine, CCI779, ABT578, a rapalog or AP23573, AP23464, AP23675 or AP23841; TAFA93, biolimus-7, biolimus-9, an ascomycin having immunosuppressive properties, e.g. ABT-281, ASM981, etc.; cyclophosphamide; methotrexate; a somatostatin analogue like octreotide, lanreotide, vapreotide or SOM230; a deoxyspergualine compound or derivative or analog thereof, e.g. 15-DSG, monoclonal antibodies to leukocyte receptors, e.g., MHC, CD2, CD3, CD4, CD7, CD8, CD11a/CD18, CD25, CD27, CD28, CD40. CD45, CD58, CD80, CD86, CD134, CD137, ICOS, CD150 (SLAM), CD152, OX40, 4-1BB or to their ligands, e.g. CD154, or antagonists thereof; other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a homologue or a mutant thereof, e.g. LEA29Y; adhesion molecule inhibitors, e.g. LFA-1 antagonists, ICAM-1 or -3 antagonists, anti-LFA-1 or anti-ICAM antibodies, VCAM-4 antagonists or VLA-4 antagonists; or anti-chemokine antibodies or anti-chemokine receptor antibodies or low molecular weight chemokine receptor antagonists, e.g. anti MCP-1 antibodies, and mixtures thereof.

25. The combination of claim 21 wherein the histone deacetylase inhibitor is selected from the group consisting of N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)-ethyl]-amino]methyl]phenyl]-2E-2-propenamide and N-hydroxy-3-[4-[[[2-(2-methyl-1H-indol-3-yl)-ethyl]-amino]methyl]phenyl]-2E-2-propenamide, or a pharmaceutically acceptable salt thereof or a pharmaceutically acceptable prodrug thereof.

26. A combination according to 21 wherein the HDAI compound and the second pharmaceutically active agent are present synergistically effective amounts.

27. A combination according to 21 wherein the HDAI compound inhibitor has an IC50 of <500 nM in the mouse or human mixed lymphocyte reaction (MLR).

28. A method of treating, preventing or suppressing an immune disorder, immune response or immune mediated response of an animal comprising administering to said animal an effective amount of an histone deacetylase inhibitor having an IC50 of <500 nM in the mouse or human mixed lymphocyte reaction (MLR).
